(19) United States
(12) Patent Application Publication
(10) Pub. No.: US 2024/0225773 A9
(43) Pub. Date: Jul. 11, 2024

(54) EYEBALL AND EYELID PROTECTOR

(71) Applicant: THE CATHOLIC UNIVERSITY OF KOREA INDUSTRY-ACADEMIC COOPERATION FOUNDATION, Seoul (KR)

(72) Inventor: Hye Young SHIN, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/191,756

(22) Filed: Mar. 28, 2023

(30) Foreign Application Priority Data
Oct. 19, 2022 (KR) 10-2022-0134718

(51) Int. Cl. A61B 90/00

(52) U.S. Cl. CPC A61B 90/04; A61B 2090/0427; A61B 2090/08021

(57) ABSTRACT

The inventive concept relates to an eyeball and eyelid protector including an eyeball cover that covers an eyeball, and an eyelid protection part that is bent and extends from one side of the eyeball cover to protect any one of an upper eyelid and a lower eyelid.

### CROSS-REFERENCE TO RELATED APPLICATIONS

A claim for priority under 35 U.S.C. § 119 is made to Korean Patent Application No. 10-2022-0134718 filed on Oct. 19, 2022 in the Korean Intellectual Property Office, the entire contents of which are hereby incorporated by reference.

### BACKGROUND

Embodiments of the inventive concept described herein relate to an eyeball and eyelid protector.

An eyelid is a wrinkled skin that covers a surface of an eyeball.

The eyelid may be classified into an upper eyelid and a lower eyelid, the upper eyelid serves to cover an upper surface of the eyeball, and the lower eyelid serves to cover a lower surface of the eyeball.

As aging of the upper eyelid and the lower eyelid progresses, various eyelid diseases may occur. In the case of the upper eyelid, blepharoptosis or blepharochalasis may frequently occur, and in the case of the lower eyelid, entropion or ectropion may frequently occur.

In the related art, to protect a cornea during upper eyelid surgery or lower eyelid surgery, an artificial implant is inserted between the upper eyelid and the cornea or between the lower eyelid and the cornea.

However, the artificial implant according to the related art may protect only the cornea and does not protect the upper eyelid or the lower eyelid. Thus, during the upper eyelid surgery or the lower eyelid surgery, the upper eyelid and the lower eyelid are not fixed, so that a surgical site is not properly exposed and parts other than the surgical site are not protected.

### SUMMARY

Embodiments of the inventive concept provide an eyeball and eyelid protector which may fix at least one of an upper eyelid and a lower eyelid during surgery of the upper eyelid or surgery of the lower eyelid.

The aspects of the inventive concept are not limited to the aspects described above, and those skilled in the art will clearly understand other aspects not described from the following description.

According to an embodiment, an eyeball and eyelid protector includes an eyeball cover that covers an eyeball, and an eyelid protection part that is bent and extends from one side of the eyeball cover to protect any one of an upper eyelid and a lower eyelid.

Further, the eyelid protection part may include an eyelid insertion portion extending from the one side of the eyeball cover to an inside of the eyeball cover and inserted into any one of a gap between the upper eyelid and the eyeball or a gap between the lower eyelid and the eyeball, an eyelid accommodation portion bent and extending from the eyelid insertion portion to an outside of the eyeball cover to accommodate any one of the upper eyelid or the lower eyelid, and an eyelash cover portion extending from the eyelid accommodation portion to the outside of the eyeball cover to cover any one of an eyelash of the upper eyelid or an eyelash of the lower eyelid.

Further, an inner surface of the eyelid insertion portion may have a smaller radius of curvature than that of an inner surface of the eyelash cover portion, and an inner surface of the eyelid accommodation portion may have a smaller radius of curvature than that of the inner surface of the eyelid insertion portion.

Further, a ratio of the radius of curvature of the inner surface of the eyelid insertion portion to the radius of curvature of the inner surface of the eyelash cover portion may be in a range of 1:0.4 to 1:06.

Further, the eyeball and eyelid protector may further include an auxiliary eyelid protection part bent and extending from the other side of the eyeball cover while having an interval from the eyelid protection part to protect the other one of the upper eyelid and the lower eyelid.

Further, the auxiliary eyelid protection part may include an auxiliary eyelid insertion portion extending from the other side of the eyeball cover to the inside of the eyeball cover and inserted into the other one of the gap between the upper eyelid and the eyeball or the gap between the lower eyelid and the eyeball, an auxiliary eyelid accommodation portion bent and extending from the auxiliary eyelid insertion portion to the outside of the eyeball cover to accommodate any one of the upper eyelid or the lower eyelid, and an auxiliary eyelash cover portion extending from the auxiliary eyelid accommodation portion to the outside of the eyeball cover to cover any one of the eyelash of the upper eyelid or the eyelash of the lower eyelid.

Further, an inner surface of the auxiliary eyelid insertion portion may have a smaller radius of curvature than that of an inner surface of the auxiliary eyelash cover portion, and an inner surface of the auxiliary eyelid accommodation portion may have a smaller radius of curvature than that of the inner surface of the auxiliary eyelid insertion portion.

Further, a ratio of the radius of curvature of the inner surface of the auxiliary eyelid insertion portion to the radius of curvature of the inner surface of the auxiliary eyelash cover portion may be in a range of 1:0.4 to 1:06.

Further, an eyeball insertion portion having a smaller thickness than those of the other portions may be formed in a circumference of the eyeball cover.

Further, the eyeball cover may have a dome-shaped cross-sectional shape.

The specific details of the inventive concept are included in the detailed description and the drawings.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a perspective view illustrating an eyeball and eyelid protector according to a first embodiment of the inventive concept;

FIG. 2 is a cross-sectional view illustrating the eyeball and eyelid protector according to the first embodiment of the inventive concept;

FIG. 3 is a cross-sectional view illustrating a state in which the eyeball and eyelid protector according to the first embodiment of the inventive concept protects an upper eyelid;

FIG. 4 is a cross-sectional view illustrating a state in which the eyeball and eyelid protector according to the first embodiment of the inventive concept protects a lower eyelid;

FIG. 5 is a perspective view illustrating an eyeball and eyelid protector according to a second embodiment of the inventive concept;

FIG. 6 is a cross-sectional view illustrating the eyeball and eyelid protector according to the second embodiment of the inventive concept; and

FIG. 7 is a cross-sectional view illustrating a state in which the eyeball and eyelid protector according to the second embodiment of the inventive concept protects an upper eyelid and a lower eyelid.

### DETAILED DESCRIPTION

Advantages and features of the inventive concept and a method of achieving the advantages and the features will become apparent with reference to embodiments described below in detail in conjunction with the accompanying drawings. However, the inventive concept is not limited to the embodiments described below but may be implemented in various forms, and the present embodiments merely make the disclosure of the inventive concept complete and are provided to completely inform the scope of the inventive concept to those skilled in the art to which the inventive concept belongs, and the inventive concept is merely defined by the scope of the appended claims.

Terms used in the present specification are intended to describe the embodiments and are not intended to limit the inventive concept. In the present specification, a singular form also includes a plural form unless specifically mentioned in a phrase. The terms “comprises” and/or “comprising” used in the specification do not exclude the presence or addition of one or more other components other than the mentioned components. Throughout the specification, the same reference numerals refer to the same components, and the term “and/or” includes each and all combinations of one or more of components mentioned. Although “first,” “second,” and the like are used to describe various components, it is apparent that these components are not limited by these terms. These terms are only used to distinguish one component from another component. Thus, it is apparent that a first component mentioned below may be a second component within the technical spirit of the inventive concept.

Unless otherwise defined, all the terms (including technical and scientific terms) used herein may be used as meanings that may be commonly understood by those skilled in the art to which the inventive concept belongs. Further, terms defined in a commonly used dictionary are not interpreted ideally or excessively unless explicitly and specifically defined.

Prior to the description of the inventive concept, in various embodiments, in the first embodiment, components having the same configuration are representatively described using the same reference numerals, and in other embodiments, a configuration different from that of the first embodiment will be described.

Hereinafter, embodiments of the inventive concept will be described in detail with reference to the accompanying drawings.

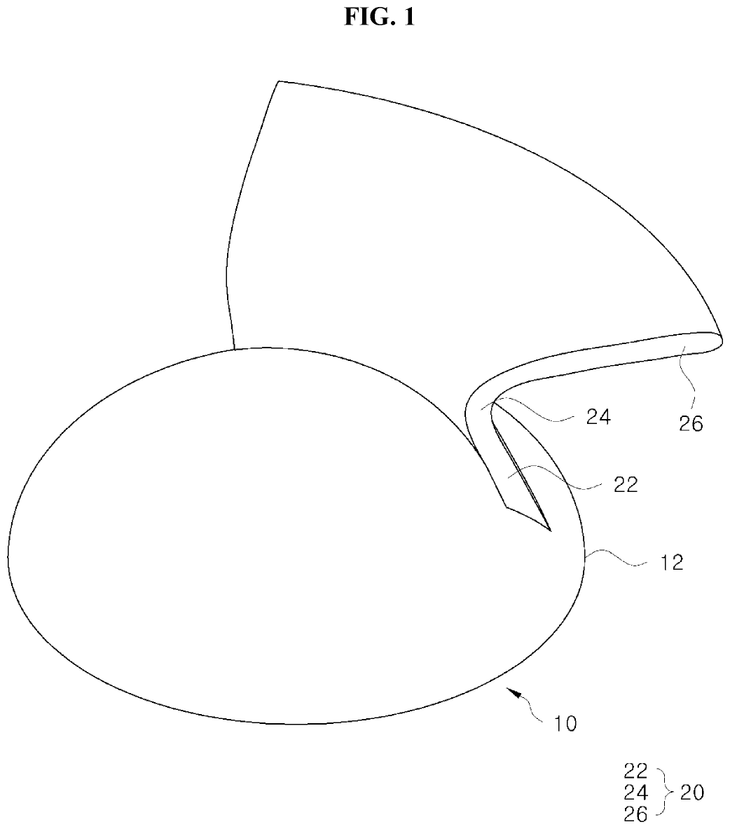

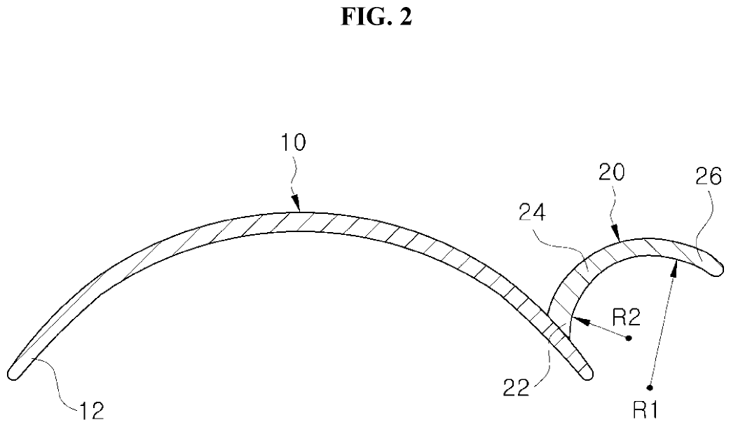

FIG. 1 is a perspective view illustrating an eyeball and eyelid protector according to a first embodiment of the inventive concept, and FIG. 2 is a cross-sectional view illustrating the eyeball and eyelid protector according to the first embodiment of the inventive concept.

As illustrated in FIG. 1, an eyeball and eyelid protector according to the first embodiment of the inventive concept may include an eyeball cover 10 and an eyelid protection part 20.

The eyeball cover 10 is inserted between an upper eyelid 2 and an eyeball 1 and between a lower eyelid 3 and the eyeball 1 during surgery of the upper eyelid 2 or surgery of the lower eyelid 3 and serves to cover the eyeball 1. Thus, during the surgery of the upper eyelid 2 or the surgery of the lower eyelid 3, the eyeball 1 is covered by the eyeball cover 10, and thus the eyeball 1 may be prevented from being damaged by a surgical tool or a surgical drug.

In an embodiment, the eyeball cover 10 may have a dome-shaped cross-sectional shape to have a shape corresponding to the eyeball 1.

In another embodiment, the eyeball cover 10 may be individually manufactured in a dome-shaped cross-sectional shape corresponding to the specific eyeball 1 by three-dimension (3D) printing.

In still another embodiment, an eyeball insertion portion 12 having a thickness smaller than those of other parts may be formed around the eyeball cover 10. The eyeball insertion portion 12 may serve to easily insert the eyeball cover 10 between the upper eyelid 2 and the eyeball 1 and between the lower eyelid 3 and the eyeball 1. Furthermore, the eyeball insertion portion 12 may have a thickness that is gradually reduced as it goes in a radial direction of the eyeball cover 10.

The eyelid protection part 20 may be bent and extend from one side of the eyeball cover 10 to protect any one of the upper eyelid 2 or the lower eyelid 3. For example, during the surgery of the lower eyelid 3, the eyelid protection part 20 may be disposed at a point, at which the upper eyelid 2 is protected, to space the upper eyelid 2 and the lower eyelid 3 apart from each other. Further, during the surgery of the upper eyelid 2, the eyelid protection part 20 may be disposed at a point, at which the lower eyelid 3 is protected, to space the lower eyelid 3 and the upper eyelid 2 apart from each other.

In an embodiment, the eyelid protection part 20 may have a thickness of 0.4 mm to 0.8 mm. However, when the thickness of the eyelid protection part 20 is less than 0.4 mm, the eyelid protection part 20 may not protect the upper eyelid 2 or the lower eyelid 3 and may be bent, and when the thickness of the eyelid protection part 20 is greater than 0.8 mm, a weight of the eyelid protection part 20 may be excessive. Thus, the thickness of the eyelid protection part 20 may be limited to the above-described value.

In another embodiment, the total height of the eyelid protection part 20 may be in a range of 4 mm to 8 mm, and in detail, the total height of the eyelid protection part 20 may be 4 mm, 6 mm, and 8 mm.

The eyelid protection part 20 may include an eyelid insertion portion 22, an eyelid accommodation portion 24, and an eyelash cover portion 26.

The eyelid insertion portion 22 may extend from the one side of the eyeball cover 10 to an inside of the eyeball cover 10 and may be inserted into any one of a gap between the upper eyelid 2 and the eyeball 1 or a gap between the lower eyelid 3 and the eyeball 1. For example, during the surgery of the lower eyelid 3, the eyelid insertion portion 22 may be inserted between the upper eyelid 2 and the eyeball 1. Further, during the surgery of the upper eyelid 2, the eyelid insertion portion 22 may be inserted between the lower eyelid 3 and the eyeball 1.

The eyelid accommodation portion 24 may be bent and extend from the eyelid insertion portion 22 to an outside of the eyeball cover 10 to accommodate any one of the upper eyelid 2 or the lower eyelid 3. For example, during the surgery of the lower eyelid 3, the eyelid accommodation portion 24 may accommodate the upper eyelid 2. Further, during the surgery of the upper eyelid 2, the eyelid insertion portion 22 may accommodate the lower eyelid 3.

The eyelash cover portion 26 may extend from the eyelid accommodation portion 24 to the outside of the eyeball cover 10 to cover any one of an eyelash of the upper eyelid 2 or an eyelash of the lower eyelid 3. For example, during the surgery of the lower eyelid 3, the eyelash cover portion 26 may cover the eyelash of the upper eyelid 2. Further, during the surgery of the upper eyelid 2, the eyelash cover portion 26 may cover the eyelash of the lower eyelid 3.

In an embodiment, an inner surface of the eyelid insertion portion 22, an inner surface of the eyelid accommodation portion 24, and an inner surface of the eyelash cover portion 26 may have a cross-sectional shape of a circular arc having a specific radius of curvature in a radial direction of the eyeball cover 10.

In detail, as illustrated in FIG. 2, the inner surface of the eyelid insertion portion 22 may have a smaller radius of curvature than that of the inner surface of the eyelash cover portion 26, and the inner surface of the eyelid accommodation portion 24 may have a smaller radius of curvature than that of the inner surface of the eyelid insertion portion 22. Here, a ratio of the radius R2 of curvature of the inner surface of the eyelid insertion portion 22 to the radius R1 of curvature of the inner surface of the eyelash cover portion 26 may be in a range of 1:0.4 to 1:0.6.

However, when the ratio of the radius R2 of curvature of the inner surface of the eyelid insertion portion 22 to the radius R1 of curvature of the inner surface of the eyelash cover portion 26 is less than 1:0.4, since the radius R2 of curvature of the eyelid insertion portion 22 is too small, an interval between the eyelid insertion portion 22 and the eyeball cover 10 becomes very small, and thus manufacturing is difficult. When the ratio of the radius R2 of curvature of the inner surface of the eyelid insertion portion 22 to the radius R1 of curvature of the inner surface of the eyelash cover portion 26 is greater than 1:0.6, the radius R2 of curvature of the eyelid insertion portion 22 is too large, and thus the eyelid insertion portion 22 may not be inserted between the upper eyelid 2 and the eyeball 1 or between the lower eyelid 3 and the eyeball 1. Thus, the ratio of the radius R2 of curvature of the inner surface of the eyelid insertion portion 22 to the radius R1 of curvature of the inner surface of the eyelash cover portion 26 may be limited to the above-described value.

Hereinafter, a process of protecting the upper eyelid 2 or protecting the lower eyelid 3 by the eyeball and eyelid protector according to the first embodiment of the inventive concept will be described.

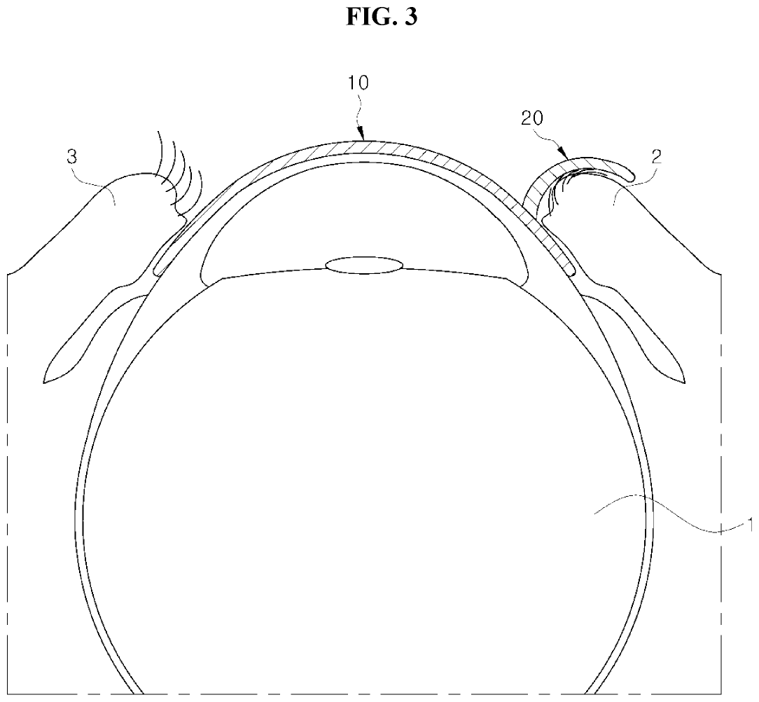

FIG. 3 is a cross-sectional view illustrating a state in which the eyeball and eyelid protector according to the first embodiment of the inventive concept protects the upper eyelid 2.

Referring to FIG. 3, a process of protecting the upper eyelid 2 by the eyeball and eyelid protector according to the first embodiment of the inventive concept will be described. The following process may be performed by a robot arm under the control of a controller or by an operator.

First, the eyelid protection part 20 moves to a position adjacent to the upper eyelid 2.

Next, an upper portion of the eyeball cover 10 is inserted between the upper eyelid 2 and the eyeball 1, and a lower portion of the eyeball cover 10 is inserted between the lower eyelid 3 and the eyeball 1. In this case, the eyeball cover 10 covers the eyeball 1. At the same time, the eyelid protection part 20 protects the upper eyelid 2.

Thereafter, the robot arm or the operator may perform the surgery of the lower eyelid 3. Here, the surgery of the lower eyelid 3 may be a process of cutting and removing a skin protrusion portion of the lower eyelid 3. In detail, the surgery of the lower eyelid 3 may be a process of cutting and removing a fat layer accommodated in the skin protrusion portion of the lower eyelid 3.

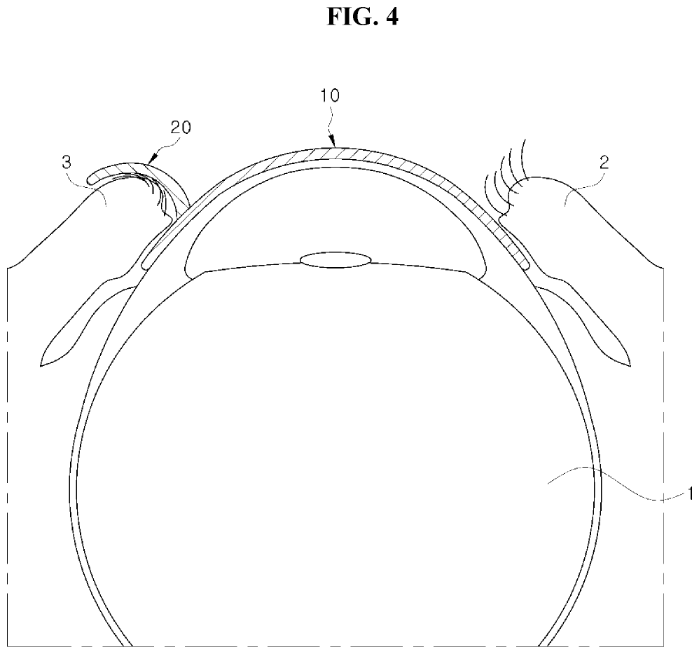

FIG. 4 is a cross-sectional view illustrating a state in which the eyeball and eyelid protector according to the first embodiment of the inventive concept protects the lower eyelid 3.

Referring to FIG. 4, a process of protecting the lower eyelid 3 by the eyeball and eyelid protector according to the first embodiment of the inventive concept will be described. The following process may be performed by the robot arm under the control of the controller or by the operator.

First, the eyelid protection part 20 moves to a position adjacent to the lower eyelid 3.

Next, the upper portion of the eyeball cover 10 is inserted between the upper eyelid 2 and the eyeball 1, and the lower portion of the eyeball cover 10 is inserted between the lower eyelid 3 and the eyeball 1. In this case, the eyeball cover 10 covers the eyeball 1. At the same time, the eyelid protection part 20 protects the lower eyelid 3.

Thereafter, the robot arm or the operator may perform the surgery of the upper eyelid 2. Here, the surgery of the upper eyelid 2 may be a process of removing a skin sagging portion of the upper eyelid 2. In detail, the surgery of the upper eyelid 2 may be a process of cutting and removing the skin sagging portion of the upper eyelid 2 and then suturing the cut portion.

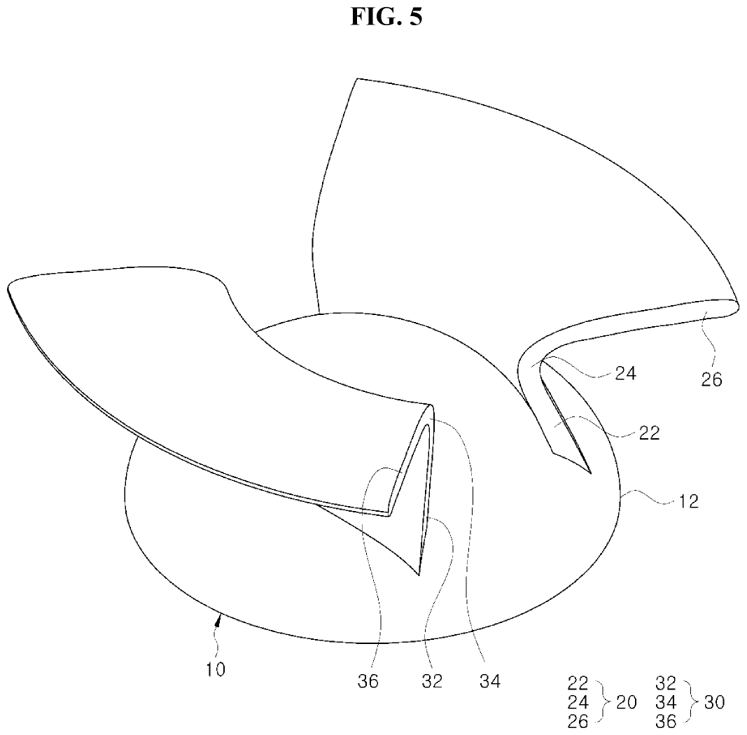

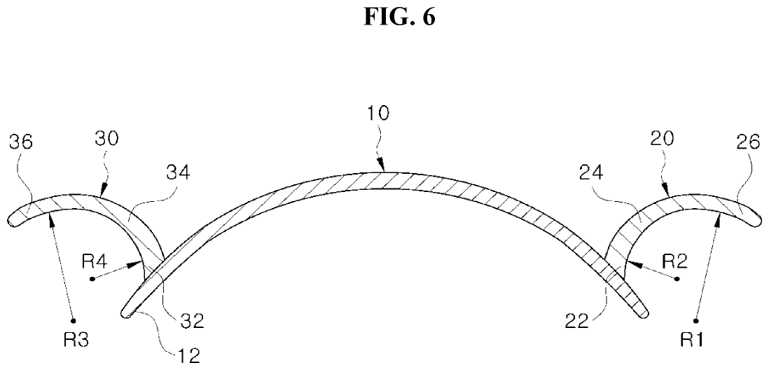

FIG. 5 is a perspective view illustrating an eyeball and eyelid protector according to a second embodiment of the inventive concept, and FIG. 6 is a cross-sectional view illustrating the eyeball and eyelid protector according to the second embodiment of the inventive concept.

As illustrated in FIGS. 5 and 6, unlike the first embodiment, the eyeball and eyelid protector according to the second embodiment may further include an auxiliary eyelid protection part 30.

The auxiliary eyelid protection part 30 may be bent and extend from the one side of the eyeball cover 10 while having an interval from the eyelid protection part 20 to protect the other one of the upper eyelid 2 and the lower eyelid 3.

Thus, in the eyeball and eyelid protector according to the present embodiment, the upper eyelid 2 and the lower eyelid 3 may be protected together by the eyelid protection part 20 and the auxiliary eyelid protection part 30. Thus, when the eyeball and eyelid protector according to the present embodiment is used, the upper eyelid 2 and the lower eyelid 3 may be protected together by the eyelid protection part 20 and the auxiliary eyelid protection part 30, and thus the surgery of the upper eyelid 2 and the surgery of the lower eyelid 3 may be simultaneously performed.

In an embodiment, the auxiliary eyelid protection part 30 may have a thickness of 0.4 mm to 0.8 mm. However, when the thickness of the auxiliary eyelid protection part 30 is less than 0.4 mm, the auxiliary eyelid protection part 30 may not protect the upper eyelid 2 or the lower eyelid 3 and may be bent, and when the thickness of the auxiliary eyelid protection part 30 is greater than 0.8 mm, a weight of the auxiliary eyelid protection part 30 may be excessive. Thus, the thickness of the auxiliary eyelid protection part 30 may be limited to the above-described value.

The auxiliary eyelid protection part 30 may include an auxiliary eyelid insertion portion 32, an auxiliary eyelid accommodation portion 34, and an auxiliary eyelash cover portion 36.

The auxiliary eyelid insertion portion 32 may extend from the one side of the eyeball cover 10 to an inside of the eyeball cover 10 and may be inserted into the other one of the gap between the upper eyelid 2 and the eyeball 1 or the gap between the lower eyelid 3 and the eyeball 1. Thus, in the present embodiment, the eyelid insertion portion 22 and the auxiliary eyelid insertion portion 32 may be inserted between the upper eyelid 2 and the eyeball 1 and between the lower eyelid 3 and the eyeball 1. Alternatively, in the present embodiment, the eyelid insertion portion 22 and the auxiliary eyelid insertion portion 32 may be inserted between the lower eyelid 3 and the eyeball 1 and between the upper eyelid 2 and the eyeball 1.

The auxiliary eyelid accommodation portion 34 may be bent and extend from the auxiliary eyelid insertion portion 32 to the outside of the eyeball cover 10 to accommodate the other one of the upper eyelid 2 or the lower eyelid 3. Thus, in the present embodiment, the eyelid accommodation portion 24 and the auxiliary eyelid accommodation portion 34 may accommodate the upper eyelid 2 and the lower eyelid 3, respectively. Alternatively, in the present embodiment, the eyelid accommodation portion 24 and the auxiliary eyelid accommodation portion 34 may accommodate the lower eyelid 3 and the upper eyelid 2, respectively.

The auxiliary eyelash cover portion 36 may extend from the auxiliary eyelid accommodation portion 34 to the outside of the eyeball cover 10 to cover the other one of the eyelash of the upper eyelid 2 or the eyelash of the lower eyelid 3. Thus, in the present embodiment, the eyelash cover portion 26 and the auxiliary eyelash cover portion 36 may cover the eyelash of the upper eyelid 2 and the eyelash of the lower eyelid 3, respectively. Alternatively, in the present embodiment, the eyelash cover portion 26 and the auxiliary eyelash cover portion 36 may cover the eyelash of the lower eyelid 3 and the eyelash of the upper eyelid 2, respectively.

In an embodiment, an inner surface of the auxiliary eyelid insertion portion 32, an inner surface of the auxiliary eyelid accommodation portion 34, and an inner surface of the auxiliary eyelash cover portion 36 may have a cross-sectional shape of a circular arc having a specific radius of curvature in the radial direction of the eyeball cover 10.

In detail, as illustrated in FIG. 6, the inner surface of the auxiliary eyelid insertion portion 32 may have a smaller radius of curvature than that of the inner surface of the auxiliary eyelash cover portion 36, and the inner surface of the auxiliary eyelid accommodation portion 34 may have a smaller radius of curvature than that of the inner surface of the auxiliary eyelid insertion portion 32. Here, a ratio of the radius R4 of curvature of the inner surface of the auxiliary eyelid insertion portion 32 to the radius R3 of curvature of the inner surface of the auxiliary eyelash cover portion 36 may be in a range of 1:0.4 to 1:0.6.

However, when the ratio of the radius R4 of curvature of the inner surface of the auxiliary eyelid insertion portion 32 to the radius R3 of curvature of the inner surface of the auxiliary eyelash cover portion 36 is less than 1:0.4, since the radius R3 of curvature of the auxiliary eyelid insertion portion 32 is too small, an interval between the auxiliary eyelid insertion portion 32 and the eyeball cover 10 becomes very small, and thus manufacturing is difficult. When the ratio of the radius R4 of curvature of the inner surface of the auxiliary eyelid insertion portion 32 to the radius R3 of curvature of the inner surface of the auxiliary eyelash cover portion 36 is greater than 1:0.6, the radius R3 of curvature of the auxiliary eyelid insertion portion 32 is too large, and thus the auxiliary eyelid insertion portion 32 may not be inserted between the upper eyelid 2 and the eyeball 1 or between the lower eyelid 3 and the eyeball 1. Thus, the ratio of the radius R4 of curvature of the inner surface of the auxiliary eyelid insertion portion 32 to the radius R3 of curvature of the inner surface of the auxiliary eyelash cover portion 36 may be limited to the above-described value.

Hereinafter, a process of protecting the upper eyelid 2 and the lower eyelid 3 by the eyeball and eyelid protector according to the second embodiment of the inventive concept will be described.

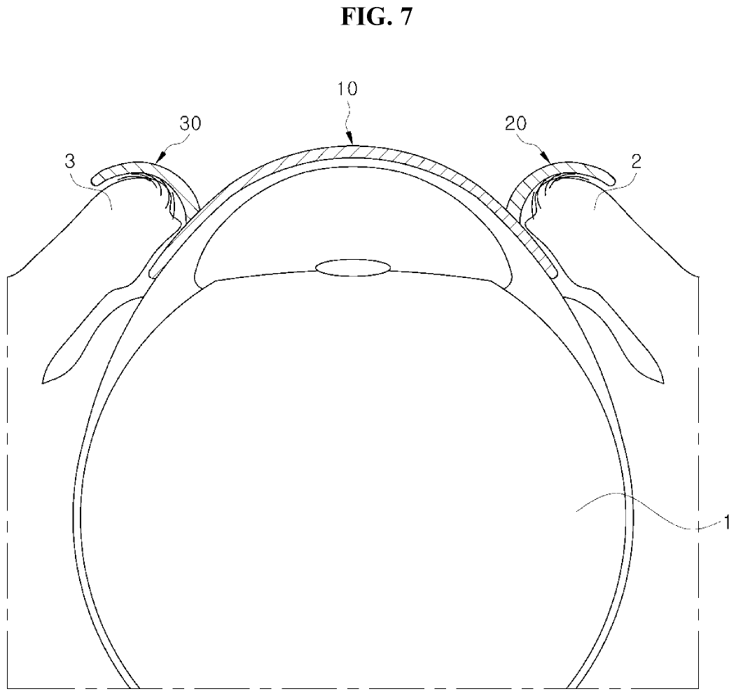

FIG. 7 is a cross-sectional view illustrating a state in which the eyeball and eyelid protector according to the first embodiment of the inventive concept protects the upper eyelid 2 and the lower eyelid 3. The following process may be performed by the robot arm under the control of the controller or by the operator.

First, the eyelid protection part 20 moves to a position adjacent to the upper eyelid 2, and the auxiliary eyelid protection part 30 moves to a position adjacent to the lower eyelid 3.

Next, the upper portion of the eyeball cover 10 is inserted between the upper eyelid 2 and the eyeball 1, and the lower portion of the eyeball cover 10 is inserted between the lower eyelid 3 and the eyeball 1. In this case, the eyeball cover 10 covers the eyeball 1. At the same time, the eyelid protection part 20 may protect the upper eyelid 3, and the auxiliary eyelid protection part 30 may protect the lower eyelid 2.

Thereafter, the robot arm or the operator may simultaneously perform the surgery of the upper eyelid 2 and the surgery of the lower eyelid 3.

According to the inventive concept, the eyeball and eyelid protector according to various embodiments of the inventive concept may fix at least one of the upper eyelid 2 and the lower eyelid 3 during the surgery of the upper eyelid 2 or the surgery of the lower eyelid 3.

Hereinabove, embodiments of the inventive concept have been described with reference to the accompanying drawings. However, those skilled in the art to which the inventive concept pertains can understand that the inventive concept can be implemented in other specific forms without changing the technical spirit or essential features thereof. Therefore, it should be understood that the embodiments described above are illustrative but not limiting in all aspects.

What is claimed is:

1. An eyeball and eyelid protector comprising:
an eyeball cover configured to cover an eyeball; and
an eyelid protection part that is bent and extends from one side of the eyeball cover to protect any one of an upper eyelid and a lower eyelid.

2. The eyeball and eyelid protector of claim 1, wherein the eyelid protection part includes:
an eyelid insertion portion extending from the one side of the eyeball cover to an inside of the eyeball cover and inserted into any one of a gap between the upper eyelid and the eyeball or a gap between the lower eyelid and the eyeball;
an eyelid accommodation portion bent and extending from the eyelid insertion portion to an outside of the eyeball cover to accommodate any one of the upper eyelid or the lower eyelid; and
an eyelash cover portion extending from the eyelid accommodation portion to the outside of the eyeball cover to cover any one of an eyelash of the upper eyelid or an eyelash of the lower eyelid.

3. The eyeball and eyelid protector of claim 2, wherein an inner surface of the eyelid insertion portion has a smaller radius of curvature than that of an inner surface of the eyelash cover portion, and
an inner surface of the eyelid accommodation portion has a smaller radius of curvature than that of the inner surface of the eyelid insertion portion.

4. The eyeball and eyelid protector of claim 3, wherein a ratio of the radius of curvature of the inner surface of the eyelid insertion portion to the radius of curvature of the inner surface of the eyelash cover portion is in a range of 1:0.4 to 1:0.6.

5. The eyeball and eyelid protector of claim 2, further comprising:
an auxiliary eyelid protection part bent and extending from the other side of the eyeball cover while having an interval from the eyelid protection part to protect the other one of the upper eyelid or the lower eyelid.

6. The eyeball and eyelid protector of claim 5, wherein the auxiliary eyelid protection part includes:
an auxiliary eyelid insertion portion extending from the other side of the eyeball cover to the inside of the eyeball cover and inserted into the other one of the gap between the upper eyelid and the eyeball or the gap between the lower eyelid and the eyeball;
an auxiliary eyelid accommodation portion bent and extending from the auxiliary eyelid insertion portion to the outside of the eyeball cover to accommodate any one of the upper eyelid or the lower eyelid; and
an auxiliary eyelash cover portion extending from the auxiliary eyelid accommodation portion to the outside of the eyeball cover to cover any one of the eyelash of the upper eyelid or the eyelash of the lower eyelid.

7. The eyeball and eyelid protector of claim 6, wherein an inner surface of the auxiliary eyelid insertion portion has a smaller radius of curvature than that of an inner surface of the auxiliary eyelash cover portion, and
an inner surface of the auxiliary eyelid accommodation portion has a smaller radius of curvature than that of the inner surface of the auxiliary eyelid insertion portion.

8. The eyeball and eyelid protector of claim 7, wherein a ratio of the radius of curvature of the inner surface of the auxiliary eyelid insertion portion to the radius of curvature of the inner surface of the auxiliary eyelash cover portion is in a range of 1:0.4 to 1:06.

9. The eyeball and eyelid protector of claim 1, wherein an eyeball insertion portion having a smaller thickness than those of the other portions is formed in a circumference of the eyeball cover.

10. The eyeball and eyelid protector of claim 1, wherein the eyeball cover has a dome-shaped cross-sectional shape.
